(19) United States
(12) Patent Application Publication
(10) Pub. No.: US 2026/0076729 A1
(43) Pub. Date: Mar. 19, 2026

(54) ABLATION SYSTEMS WITH REDUCED ENERGY LOSS

(71) Applicant: Venclose, Inc., San Jose, CA (US)

(72) Inventors: Darius Przygoda, Mountain View, CA (US); Negin Behzadian, Mesa, AZ (US)

(21) Appl. No.: 18/887,444

(22) Filed: Sep. 17, 2024

(51) Int. Cl. A61B 18/08; A61B 17/00; A61B 18/00

(52) U.S. Cl. CPC A61B 18/082; A61B 2017/00199; A61B 2017/00292; A61B 2018/00404; A61B 2018/00577; A61B 2018/00714; A61B 2018/00797; A61B 2018/0091

(57) ABSTRACT

In some examples, a power routing circuit of an endovascular ablation system may include components with reduced forward voltage drops in a handle of a thermal ablation catheter. The handle may also include a reference temperature sensor used for closed-loop temperature control. In some examples, power routing circuit may include transistors such as metal-oxide-semiconductor field-effect transistors (MOSFETs). In some examples, transistors may be coupled in parallel with diodes in the power routing circuit. In some examples, the diodes may be Schottky diodes. The reduction in forward voltage drops may reduce heat dissipation of the power routing circuit, which may reduce heating of the reference temperature sensor. Power losses in the handle that lead to reduced power in the distal heating elements may be reduced.

### BACKGROUND

Blood vessels and other physiological structures can fail to perform their proper function. An example, in the case where opposing valve leaflets within a vein do not touch each other, blood flow within the vein is not predominately restricted to one direction towards the heart. This condition is called venous reflux, and it causes elevated localized blood pressure within the vein. Elevated localized blood pressure is subsequently transferred to the surrounding tissue and skin.

Endovenous thermal ablation is a technique where heat is applied within the vein to cause the vein wall to permanently shrink to the point the vein lumen is occluded. A portion of a catheter is inserted into a vein, and power is delivered to heating elements located in the inserted portion of the catheter to apply the heat. The amount of heat applied to the vein is based, at least in part, on the power delivered to the heating elements. A control system measures temperature at or near the heating elements. Based on the measured temperature, the control system may increase or decrease the amount of power provided to the heating elements in order to maintain the heating elements at a desired temperature (or within a desired temperature range). If the heating elements are not raised to a sufficient temperature (e.g., 120° C.) for a sufficient time, the vein wall may not shrink and occlude the vein lumen. If the heating elements are raised to a temperature that is too high, surrounding tissue may be damaged. Accordingly, accurate temperature measurements are desirable.

### BRIEF DESCRIPTION OF THE DRAWINGS

FIG. 1 depicts a diagram of an example of a system for providing endovascular thermal ablation according to at least one embodiment of the present disclosure.

FIG. 2 depicts a diagram of a catheter for providing endovascular thermal ablation according to at least one embodiment of the present disclosure.

FIG. 3 depicts a block diagram of circuitry included in a handle of a catheter according to at least one embodiment of the present disclosure.

FIG. 4 depicts a block diagram of heating elements and components of a power routing circuit of a catheter according to at least one embodiment of the present disclosure.

FIG. 5 depicts a circuit diagram of components of a power routing circuit of a catheter according to at least one embodiment of the present disclosure.

FIG. 6 depicts a circuit diagram of components of a power routing circuit of a catheter according to at least one embodiment of the present disclosure.

FIG. 7 depicts a flow chart of a method according to at least one embodiment of the present disclosure.

### SUMMARY

According to an embodiment, a system for venous ablation may include a catheter (e.g., 102) including a tube (e.g., 228) including a first heating element (e.g., 223) and a second heating element (e.g., 225) coupled in series between a first terminal (e.g., terminal B) and a second terminal (e.g., terminal A), and a power routing circuit (e.g., 330) including: a first transistor (e.g., 542) having a first node coupled between the first heating element and the second heating element and a second node coupled to the first terminal of the catheter and a second transistor (e.g., 544) having a third node coupled to the second heating element and a fourth node coupled to the first terminal of the catheter, wherein the first transistor includes a different type than the second transistor.

In some embodiments, the first transistor includes an N-channel metal-oxide semiconductor field-effect transistor (MOSFET) transistor and the second transistor includes a P-channel MOSFET transistor. In some embodiments, a gate of the first transistor and a gate of the second transistor are coupled to the second terminal (e.g., terminal A).

In some embodiments, the power routing circuitry may further include a first diode (e.g., 440) coupled in parallel with the first transistor and a second diode (e.g., 438) coupled in parallel with the second transistor, wherein the first diode is configured to conduct in a first direction and the second diode is configured to conduct in a second direction different than the first direction.

In some embodiments, the catheter further includes a handle coupled to the tube, wherein the power routing circuit is included in the handle. The tube may further includes a first temperature sensor, and the handle includes a second temperature sensor. In some embodiments, the system may include an energy delivery console configured to provide a voltage to the first terminal and the second terminal.

According to an embodiment, a catheter for venous ablation may include a first transistor (e.g., 542) having a first node coupled between a first resistance (e.g., 223) and a second resistance (e.g., 225) and a second node coupled to a first terminal (e.g., terminal B) and a second transistor (e.g., 544) having a third node coupled to the second resistance and a fourth node coupled to the first terminal, wherein the first transistor includes a different type than the second transistor. In some embodiments, the catheter may further include a first diode (e.g., 440) coupled in parallel with the first transistor and a second diode (e.g., 438) coupled in parallel with the second transistor.

According to an embodiment, a method of controlling first and second heating elements of a system for venous ablation may include applying a voltage in a first polarity between a first terminal and a second terminal (e.g., terminals A and B), passing a current through a first transistor (e.g., 542) responsive to the voltage, wherein the current is passed responsive to the voltage increasing a gate-source voltage of the first transistor, heating the first heating element (e.g., 223) responsive to the passing current, and impeding heating of the second heating element (e.g., 225) with a second transistor (e.g., 544). In some embodiments, the method may further include applying a second voltage in a second polarity opposite the first polarity between the first terminal and the second terminal, passing a second current through the second transistor responsive to the second voltage, wherein the second current is passed responsive to the second voltage increasing a gate-source voltage of the second transistor, and heating the first heating element and a second heating element responsive to the passing current. In some embodiments, the method may further include passing the current through a first diode (e.g., 440) coupled in parallel with the first resistor responsive to the voltage, wherein the current is passed responsive to the voltage meeting or exceeding a forward voltage drop of the first diode and impeding heating of the second heating element with a second diode (e.g., 438). In some embodiments, the method may further include applying a second voltage in a second polarity opposite the first polarity between the first terminal and the second terminal, passing a second current through the second transistor responsive to the second voltage, wherein the second current is passed responsive to the second voltage increasing a gate-source voltage of the second transistor, and passing the second current through a second diode responsive to the second voltage, wherein the second current is passed responsive to the voltage meeting or exceeding a forward voltage drop of the second diode.

Features from any of the disclosed embodiments may be used in combination with one another, without limitation. In addition, other features and advantages of the present disclosure will become apparent to those of ordinary skill in the art through consideration of the following detailed description and the accompanying drawings.

### DETAILED DESCRIPTION

Certain details are set forth below to provide a sufficient understanding of embodiments of the disclosure. However, it will be clear to one having skill in the art that embodiments of the disclosure may be practiced without these particular details. Moreover, the particular embodiments of the present disclosure described herein are provided by way of example and should not be used to limit the scope of the disclosure to these particular embodiments.

Various techniques may be used to accurately determine a temperature at a distal tip of a heated catheter such as an endovascular thermal ablation catheter. For example, the Seebeck Effect involves an electromotive force develops across two points of electrically conducting materials when there is a temperature difference between the materials, where one is referred to as the “hot junction” and the other is the “cold junction”. The difference of temperatures between two junctions is converted into electrical voltage, which is then used to determine a differential temperature. To accurately measure an absolute temperature at a temperature sensor at a desired point (e.g., hot junction), the absolute temperature at a second temperature sensor at the cold junction (sometimes referred to as the “cold reference junction temperature” or “reference temperature”) must be known. Other techniques for determining a temperature at a location based, at least in part, on a temperature at another location may also be used.

A catheter may include a temperature sensor at or near the heating elements. A second reference temperature sensor may be located at a distal end of the catheter or another component. For example, the reference temperature sensor may be located in a handle of the catheter or an energy delivery console. However, other electronic components in the handle or energy delivery console may dissipate heat and cause an increase in the reference temperature detected by the reference temperature sensor. This positive error in reference temperature measurements at the reference temperature sensor (e.g., the cold junction) may result in overestimating the temperature at the distal end of the catheter. This may cause a control system (e.g., in an energy delivery console) to decrease the power (e.g., by reducing a voltage) to proportionally decrease the output temperature produced at the distal tip of the catheter. This potentially compromises the efficacy of the venous ablation treatment because the catheter may not reach the temperature required to shrink vessel tissue and/or ablate the tissue.

According to embodiments of the present disclosure, a power routing circuit with reduced forward voltage drop components may be included in a handle of a thermal ablation catheter. In some examples, a power routing circuit may include transistors such as MOSFETs. In some examples, transistors may be provided in parallel with diodes in the power routing circuit. In some examples, the diodes may be Schottky diodes. The reduction in forward voltage drops may reduce heat dissipation by the power routing circuit, which may reduce heating of the reference temperature sensor (e.g., cold junction). This may improve accuracy of temperature measurements at the distal end of the catheter, helping to ensure effective treatment.

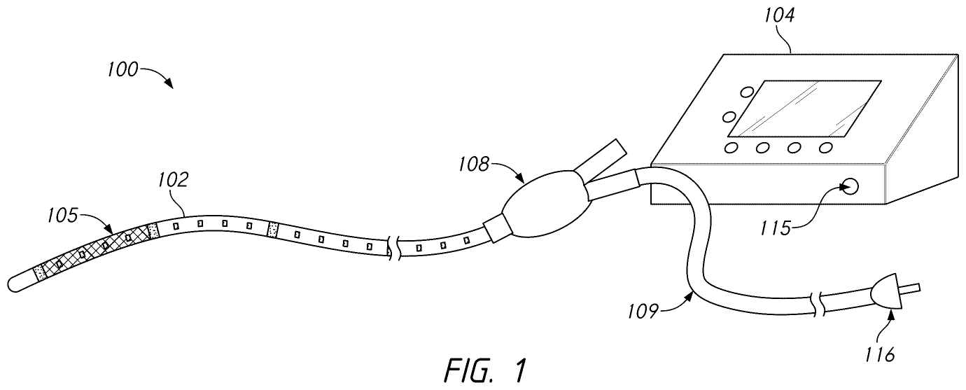

FIG. 1 depicts a diagram of an example of a system for providing endovascular thermal ablation according to at least one embodiment of the present disclosure. In this example, the system 100 includes a heated catheter 102, and an energy delivery console 104. In the illustrated example, the heated catheter 102 is a long, thin, flexible, or rigid device that can be inserted into a narrow anatomical lumen, such as a vein. Examples of systems and methods to use a heated catheter 102 to treat venous disease are described in International Application No. PCT/US2021/028779, entitled “METHODS AND SYSTEMS FOR VENOUS DISEASE TREATMENT,” which is incorporated herein by reference in for any purpose. The heated catheter 102 is connected to energy delivery console 104 to provide energy to drive one or more heating elements 105 that heat the distal end of the heated catheter 102, which can be placed within the lumen of a vein to be treated. As an example of treating venous disease, the heated catheter 102 can be inserted into a target region of a perforator vein at or below a facia layer. The heating element(s) 105 of the heated catheter 102 can be activated to provide heat therapy or heat treatment to the target region at or below the fascia layer. More than one treatment cycle can be performed in the same position within the vein before moving to the next treatment site. The heated catheter 102 can be moved (e.g., withdrawn proximally towards the entry point through the skin of the patient) to a second target region within, for example, the perforator vein, but still at or below the fascia layer. More heat treatments can be applied at subsequent target regions. These subsequent regions can be treated with all the heating elements 105 or a subset of the heating elements as described herein. The heated catheter 102 can be moved (e.g., retracted) to a third target region, this time above the fascia layer. Additionally, the heating element(s) 105 can be activated to provide heat therapy to the third target region above the fascia layer.

The heating elements 105 have an associated resistance causing them to heat up when electrical current passes therethrough, thereby enabling heating of the vein wall via conductive heat transfer. The heating elements 105 generate heat regardless of the direction of current flowing through them. In some examples, the energy delivery console 104 provides energy to drive one or more heating elements 105 to heat the heated catheter 102 at varying lengths along the distal end of heated catheter 102. In some examples, to power and control both longer-length and shorter-length heating elements 105, the energy delivery console 104 adjusts the power source voltage based on the lengths of the heating elements 105 to be energized. In some examples, the heating elements 105 have resistances to achieve approximately 5.7 W/cm of maximum heating, in some embodiments, the heating elements 105 have resistances to achieve approximately 50 W or below of maximum heating, which is then reduced to lower levels of heating as necessary to maintain a target temperature. Other resistances for different desired power levels may be used in other examples. This level of heating is an appropriate match for a studied protocol for thermal ablation of veins at 120° C. with a reasonably fast heating time, but alternative perturbations of higher or lower maximum heating may also be employed; examples are greater than 6 W/cm for even faster heating or to a higher temperature, or over a larger diameter heating element, or less than 5 W/cm for slower heating or to a lower temperature.

In the illustrated example, the heated catheter 102 includes a handle 108 and a connector 116. The handle 108 may include electronics that provide catheter data (e.g., memory storing a catheter identifier (ID) and catheter usage data, a temperature sensor, etc.), connections configured for the heating elements 105 of the heated catheter 102, and/or connections to a thermocouple, etc. The connector 116 provides an electrical connection between the energy delivery console 104 and the handle 108. The connector 116 plugs into corresponding plugs 115 of the energy delivery console 104. The connector 116 is configured to plug into the energy delivery console 104 in a manner that inhibits incorrectly inserting the connector 116. In some examples, the connector 116 includes two jacks of different physical configurations (e.g., a jack with a first length and diameter, and a jack with a second length and diameter, etc.). In the illustrated example, the connector 116 is electrically coupled to the handle 108 via a wire harness 109. While the connector 116 illustrated in FIG. 1 to interface with the energy delivery console 104 includes one jack, the energy delivery console 104 may be configured to be compatible with heated catheters that include two or more jacks such that the energy delivery console 104 is backwards compatible with other heated catheters.

Additional details and examples of the energy delivery console 104, heating elements 105, connector 116, and wire harness 109 may be found in International Application No. PCT/US2022/078856, entitled “SYSTEMS, HUBS, AND CONNECTOR ASSEMBLIES FOR VENOUS DISEASE TREATMENT,” and U.S. Pat. No. 10,357,305, entitled “VENOUS DISEASE TREATMENT,” which are incorporated by reference in for any purpose.

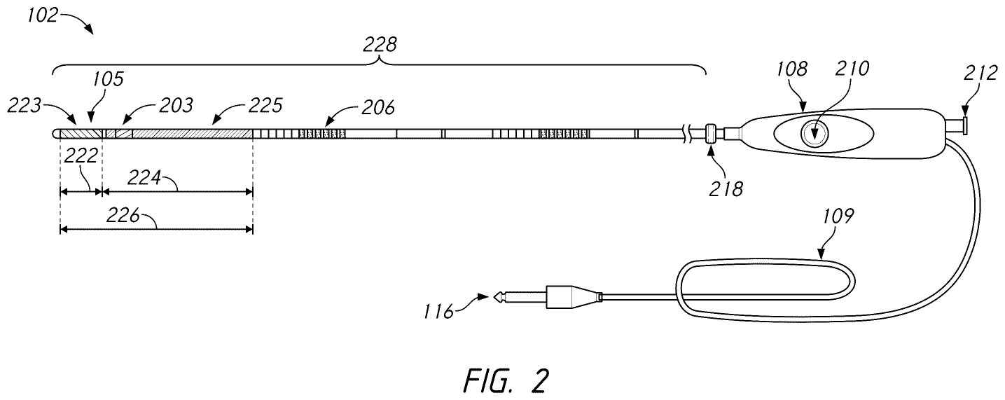

FIG. 2 depicts a diagram of a catheter for providing endovascular thermal ablation according to at least one embodiment of the present disclosure. FIG. 2 depicts the heated catheter 102 having a catheter tube 228 including one or more of the heating elements 105. The catheter tube 228 may be configured to be inserted into a vessel, such as a vein. The heating element is heated by electrical current flowing through one or more heating elements 105. In some examples, the heated catheter 102 includes multiple heating elements 105 connected in series. In some such examples, the active heating length of catheter tube 228 is configured to be selectable by the user by directing electrical current to one or more heating elements (e.g., heating coils). For example, the active heating length may be selectable from 1 cm to 10 cm. In some examples, the active heating length may be selectable between a short length and a long length. In such an example, a user (e.g., doctor, surgeon, etc.) may select a short heating length 222 (e.g., 2.5 cm) that corresponds to a first heating element 223 or a long length 226 (e.g., 10 cm) that corresponds to the first heating element 223 having a length 222 and a second heating element 225 having a length 224 (e.g., 7.5 cm), for example, by selecting a switch on heated catheter 102 or energy delivery console 104. In such an example, when a small length 222 is selected, electrical current is directed through the first heating element 223 and when full length 226 is selected, electrical current is directed through the first and second heating elements 223, 225.

The heated catheter 102 includes a temperature sensor 203 (e.g., thermocouple or thermistor) located at a position along the length of the heating elements 105, such as at a position 1-2.5 cm from the distal end of heated catheter 102. In some embodiments, a location of the temperature sensor 203 may be based, at least in part, on a length of heating element 223. The temperature sensor 203 may be placed between coil turns (with spacing or insulation to prevent electrical shorting across coils), over the coil assembly (insulated, for example, by a layer over the metal coil such as FEP, PTFE or parylene to prevent electrical shorting across coils), under the coil assembly, or within the body of heated catheter 102 under the heating element area.

In the illustrated example, markings 206 may be provided at different lengths along heated catheter 102 to guide a user by visual cues. The markings 206 may be in the form of a series of dots, spaced approximately equal to the length of the shortest heating length 222 in some embodiments. In some examples, the markings 206 are geometric lines or shapes, alphanumeric characters, color-coded features, or a combination thereof. In some examples, the markings 206 are placed at intervals approximately equal to the length of the heating element 105 (such as 10 cm apart when the heating element is 10 cm long), or slightly longer than the heating element 105 (such as 10.1 cm apart when the heating element is 10 cm long) to prevent accidental overlapping of treatments. The markings 206 may include alignment markings to facilitate location of the heating element and/or tubing bonds. In a specific implementation, a marking or discernable feature can indicate a minimal distance of treatment away from the active length of the heating element, giving the user a cue to avoid tissue heating too near the subject's skin. In one example, a marking or edge of a tubing layer or bond can be 2.5 or 3.0 cm proximal to the proximal end of the heated catheter 102.

The catheter 102 may further include a movable marker 218, which may be slide along the length of the tube 228. The marker 218 may facilitate a user advancing the heated catheter 102 a desired amount. For example, the marker 218 may be moved to align with one of the markings 206 to allow a user to advance the tube 228 a desired length.

The handle 108 may be coupled to a proximal end of the tube 228. The handle 108 may include a button 210 which communicates with the internal circuitry of the handle 108. The button 210 may start and stop delivery of energy to the heating elements 105. In some examples, such as the one shown in FIG. 2, the handle 108 may further include a luer connector 212 (cap not shown). The luer connector 212 may be coupled to the tube 228 and/or another tube (not shown) included in the heated catheter 102. The luer connector 212 may allow for fluid-tight coupling to deliver fluids (e.g., saline) along or through the heated catheter 102. However, in other examples, the luer connector 212 may be omitted.

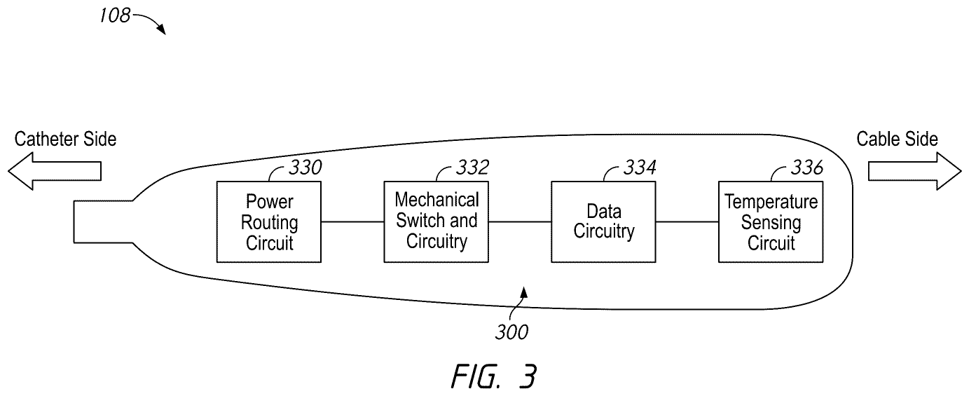

FIG. 3 depicts a block diagram of circuitry included in a handle of a catheter according to at least one embodiment of the present disclosure. The circuitry 300 of handle 108 may provide an interface between the catheter tube 228 of the heated catheter 102 and the energy delivery console 104. In some examples, the handle 108 is configured to interface with the energy delivery console 104 where circuitry to drive the heating elements 105 is located in the energy delivery console 104. In the illustrated example, the circuitry 300 includes power routing circuit 330, mechanical switch and circuitry 332 data circuitry 334, and temperature sensing circuit 336. A greater or fewer number of components may be included without departing from the scope of the present disclosure.

The data circuitry 334 may include a memory integrated circuit, such as an electrically erasable programmable read-only memory (EEPROM), that stores information related to the heated catheter 102, data from the temperature sensing circuit 336, and/or other information. For example, the data circuitry 334 may store a unique identifier for the heated catheter 102 and/or data associated with usage of the heated catheter 102 (e.g., date when it was first used, total usage time, etc.). The unique identifier may be used, for example, by the energy delivery console 104 to determine the configuration (e.g., number of coils, which terminals correspond to which of the coils, etc.) of the heated catheter 102 by, for example, comparing the unique identifier to a database for records that associate the unique identifiers to models and/or configuration settings. This database may be stored in the energy delivery console 104 may be stored in a cloud storage system accessible by the energy delivery console 104. The energy delivery console 104 then manipulates the power drivers within the energy delivery console 104, to drive the heating elements 105 of heated catheter 102 according to the arrangement of its heating elements 105 as determined by the configuration.

The temperature sensing circuit 336 may include a suitable temperature sensing component such as a thermocouple and/or thermistor. In some embodiments, the temperature detected by the temperature sensing circuit 336 may be used to facilitate determination of a temperature detected by the temperature sensor 203, for example, by using the Seebeck Effect. The difference of temperatures between the distal end of the catheter tube 202 and the handle 108 may be converted into an electrical voltage. To determine the temperature at the distal end of the catheter tube 228, the temperature at temperature sensing circuit 336 (the “cold reference junction temperature” or “reference temperature”) may be needed in order to correctly calibrate the temperature measurement in some embodiments. The temperature sensing circuit 336 may provide temperature information to the data circuitry 334 and/or directly to the energy delivery console 104. In some examples, the data circuitry 334 and temperature sensing circuit 336 may be combined, and the data circuitry 334 may include a digital temperature sensor integrated with EEPROM. The energy delivery console 104 may use the temperature information provided by the temperature sensing circuit 336 and/or data circuitry 334 to determine an amount of energy/power (e.g., by measuring current and/or voltage) to provide to the heating elements 105 to achieve the desired temperature at the treatment site.

The power routing circuit 330 may be coupled to the terminals of some of the heating elements 105. In some examples, the power routing circuit 330 connects two heating element-side terminals to one connector-side terminal and restricts the flow of current such that current can only flow in or out of one of the heating element-side terminals. By reversing the polarity of the power source on the one connector-side terminal, the energy delivery console 104 selects which of the two heating element-side terminals the current flows.

The mechanical switch and circuitry 332 may be coupled to button 210. When the button 210 is pressed, the mechanical switch and circuitry 332 may enable the power routing circuit 330 to provide power to the heating element(s) 105 with the polarity and voltage determined by the energy delivery console 104. In some embodiments, the button 210 may further be used to select the polarity of the power provided to the power routing circuit 330.

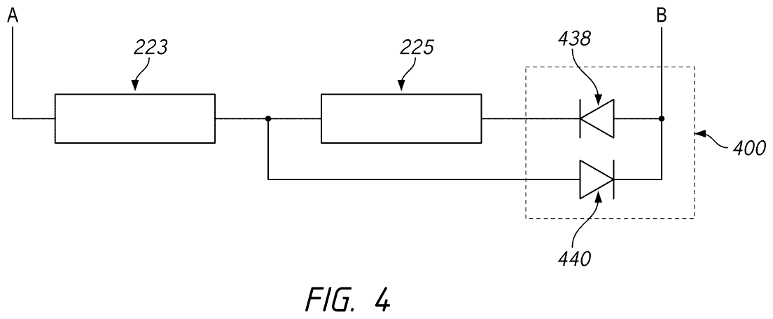

FIG. 4 depicts a block diagram of heating elements and components of a power routing circuit of a catheter according to at least one embodiment of the present disclosure. The power routing circuit components 400 may be included in power routing circuit 330 in some embodiments. The power routing circuit components 400 includes two diodes 438 and 440. Diodes 438 and 440 are power diodes. Terminals A and B may be coupled to the energy delivery console 104 (e.g., via wire harness 109 and connector 116).

The heated catheter 102 may include multiple heating elements 105, such as heating element 223 and heating element 225. The heating elements 223 and 225 are coupled in series. As discussed with reference to FIG. 2, the heated length may be length 222 or length 226. The desired length is selected by changing the polarity of the voltage energizing the heating elements 105 using diodes 438 and 440. When the voltage provided by the energy delivery console 104 is such that terminal A is positive and terminal B is negative, diode 440 allows current to flow, but diode 438 impedes the current. This causes heating element 225 to be bypassed, and only heating element 223 is heated. Thus, the heated length is length 222. When the voltage provided by the energy delivery console 104 is such that terminal A is negative and terminal B is positive, diode 440 impedes current, and diode 439 allows the current to flow. Thus, current flows through both heating element 223 and 225, and the total heated length is length 226.

Diodes need certain amount of voltage across them to start conducting (i.e., forward voltage drop, VF). The presence of this voltage causes energy loss in the diode (which is equal to VF*IF, where IF is the current flowing through the diode), which is released as heat. This heat may accumulate in the handle 108 causing a positive error in the ambient temperature measurement by temperature sensing circuit 336. Even when Schottky diodes with low VF are selected, the energy losses with the typical heating element 105 working current may result in temperature errors.

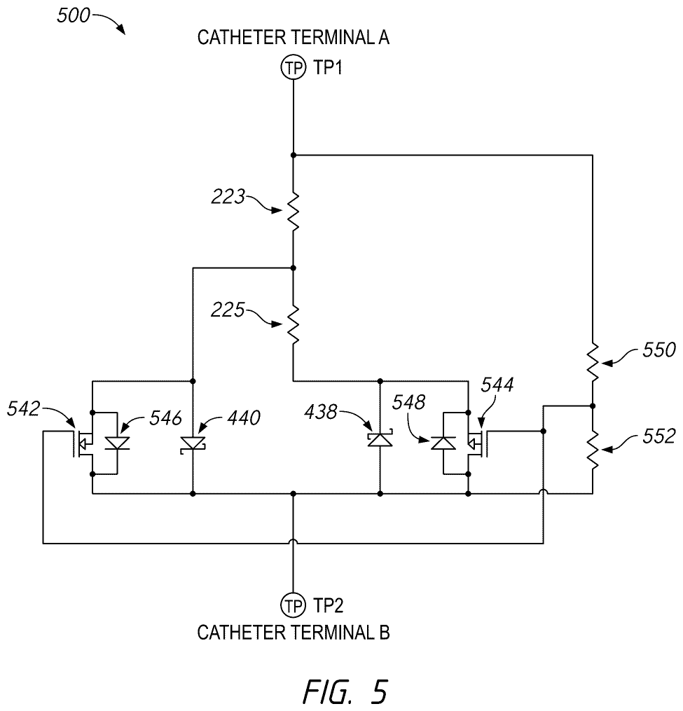

FIG. 5 depicts a circuit diagram of components of a power routing circuit of a catheter according to at least one embodiment of the present disclosure. The power routing circuit components 500 may be included in power routing circuit 330 in some embodiments. Terminals A and B may be coupled to the energy delivery console 104. The power routing circuit components 500 may include diodes 440 and diodes 438 coupled in parallel with transistors 542 and 544, respectively. In some embodiments, the power routing circuit components 500 may further include resistances 550 and 552.

For context, heating elements 223 and 225 are shown as resistances in FIG. 5. In some examples, the heating elements 223 and 225 may have low resistance (e.g., 2 Ohms and 6 Ohms, respectively). While heating elements 223 and 225 are shown, these components are included in a distal end of the catheter tube 228, and the remaining components may be located in handle 108 of the catheter 102, including terminals A and B. In some embodiments, only terminal A is directly connected to heating element 223 (e.g., via a conductive wire), and terminal B may be indirectly connected via various components.

The anode of diode 440 is coupled at a node between heating element 223 and heating element 225, and the cathode is coupled to terminal B. In some embodiments, diode 440 may be a Schottky diode. Transistor 542 may be coupled in parallel with diode 440. More specifically, a node (e.g., drain) of transistor 542 may be coupled to the node between heating element 223 and heating element 225 (and to the anode of diode 440), and another node (e.g., source) of transistor 542 may be coupled to terminal B (and to the cathode of diode 440). In some embodiments, transistor 542 may be a MOSFET. Specifically, transistor 542 may be a N-channel MOSFET. N-channel MOSFETs conduct with positive gate-to-source voltages. Transistor 542 may have low to moderately low channel resistance (e.g., mOhm range).

The cathode of diode 338 is coupled to heating element 225, and the anode is coupled to terminal B. In some embodiments, diode 338 may be a Schottky diode. Transistor 544 may be coupled in parallel with diode 438. A node (e.g., drain) of transistor 544 may be coupled to heating element 225 (and the cathode of diode 438) and another node (e.g., source) may be coupled to terminal B (and the anode of diode 438). In some embodiments, transistor 544 may be a MOSFET. Specifically, transistor 544 may be a P-channel MOSFET. P-channel MOSFETs conduct with negative gate-to-source voltages. Transistor 544 may have low to moderately low channel resistance. The gate of transistor 542 may be coupled to the gate of transistor 544. In some embodiments, the gates of transistors 542 and 544 may be coupled to terminal A. In some embodiments, a voltage provided by terminal A may drive the gates.

In an embodiment, resistance 550 may be coupled between terminal A and the gates of transistors 542 and 544, and resistance 552 may be coupled between resistance 550 and the nodes of transistors 542 and 544 and terminal B (as well as between the cathode of diode 440 and anode of diode 438). In some embodiments, the resistances 550 and 552 may be resistors. The resistance values may be based, at least in part, on the properties of the transistors 542 and 544. In some embodiments resistance 550 may be approximately 10k Ohm, and the resistance of resistance 552 may be approximately 30k Ohm. In some applications, resistances 550 and 552 may act as a resistance divider to reduce the voltage applied between the gate and source of the transistors 542 and 544.

The transistors 542 and 544 may be lower resistance than the diodes 438, 440. The transistors 542, 544 may activate when the diodes 438, 440 are conducting and bypass the diodes 438, 440. When the voltage energizing the heating elements 223, 225 is low, the transistors 542, 554 are not conducting current, and the current is flowing through either diode 440 (when terminal A is positive) or diode 438 (when terminal B is positive), depending on the voltage polarity provided by the energy delivery console 104. Since the voltage is low, the current flowing through the diode 438 or 440 is also low, so the heat dissipated in the diode 438 or 440 is small. When the energy delivery console 104 increases voltage on the terminals A and B, the absolute value of the gate-source voltage of the transistor 542 or 544 (depending on the polarity) becomes sufficiently high, saturating the transistor 542 or 544. The saturation causes the current to start flowing through the channel of transistor 542 or 544 instead of diode 440 or 438, respectively.

Due to the low channel resistance (mOhm range) of the transistors 542 and 544, the drain-source voltage across the transistors 542 or 544 is very small compared to the forward voltage of the diodes 438, 440. This lower voltage may result in lower power losses in the power routing circuit components 500 compared to power routing circuit components 400. The lower power losses in the power routing circuit components 500 may be up to an order of magnitude lower than power routing circuit 400 when the energy delivery console 104 is providing higher voltages. This may reduce power dissipation by the power routing circuit components 500, which may reduce heating in the handle 108.

Transistors 542 and 544 may include parasitic diodes, also known as “body diodes.” This is illustrated by parasitic diodes 546 and 548. However, it should be understood that parasitic diodes 546 and 548 are not separate components or additional components included in the transistors 542 and 544, but rather are inherent properties of transistors 542 and 544. In some applications, the parasitic diode properties of the transistors 542 and 544 may be used to reduce a number of components in the power routing circuit components 500.

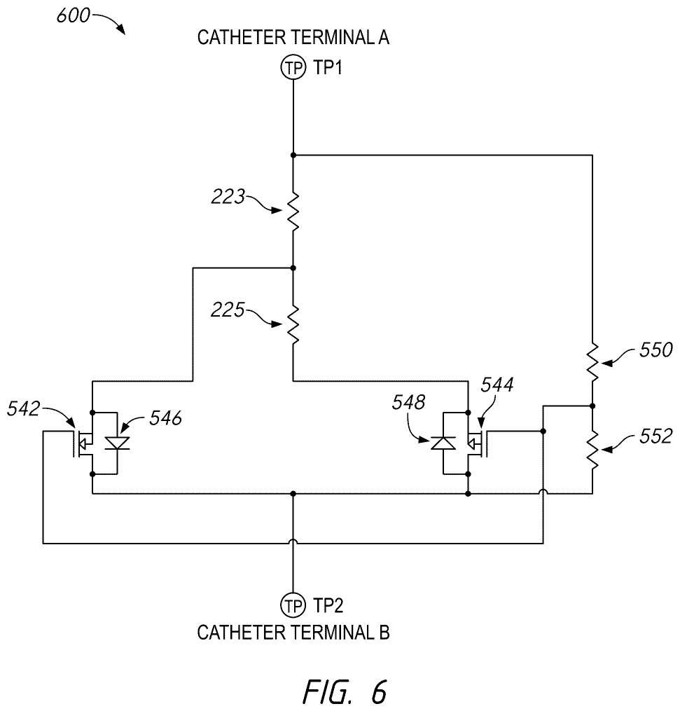

FIG. 6 depicts a circuit diagram of components of a power routing circuit of a catheter according to at least one embodiment of the present disclosure. The power routing circuit components 600 may be included in power routing circuit 330 in some embodiments. Terminals A and B may be coupled to the energy delivery console 104. The energy delivery console 104 may provide power to terminals A and B in different polarities. The power routing circuit components 500 may include transistors 542 and 544. In contrast to FIG. 5, diodes 438 and 440 are omitted, but the coupling of the remaining components may be substantially the same for power routing circuit components 600. In some embodiments, the power routing circuit components 600 may further include resistances 550 and 552. For context, heating elements 223 and 225 are shown as resistances in FIG. 6.

The parasitic diodes 546 and 548 may have sufficient current carrying capability to withstand the working current though the heating elements 223 and 225. However, the forward voltage drop VF of parasitic diodes 546 and 548 may be larger than the VF of diodes 438 and 440. The VF of diodes 438 and 440 may be 0.7V in some examples. Thus, the power dissipation for the lower voltages energizing the heating elements 223 and/or 225 may be higher through transistor 542 or 544 alone than diode 438 or 440 in parallel with the transistors 542, 544. For small voltages driving the heating elements 223, 225, the current flowing through the heating elements 223, 225 is small. Thus, the power losses in the parasitic diodes 546, 548 are an acceptable level. During normal operation, the voltage provided to the heating elements 223, 225 is typically at a low level for a short period of time before increasing. Accordingly, the transistor 542 or 544 should saturate in a short period of time, so the time over which the parasitic diode 546 or 548 may cause additional heating is also short, and the overall heating of the handle 108 may be lower than compared to power routing circuit components 400. Furthermore, during normal operation, the voltage driving the heating elements 223, 225 is not anticipated to be on the voltage level smaller than approximately 3-4V, which may also reduce the risk of excessive heating due to parasitic diodes 546 and 548.

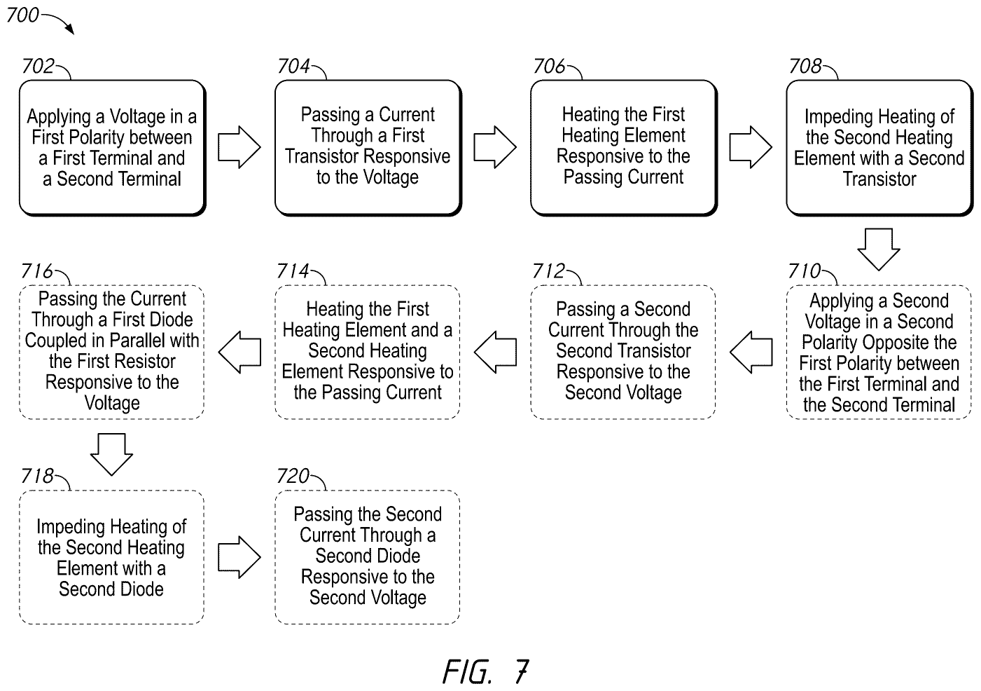

FIG. 7 depicts a flow chart of a method according to at least one embodiment of the present disclosure. The method shown in flow chart 700 is a method for controlling first and second heating elements of a medical device for venous ablation. In some embodiments, the method in flow chart 700 may be performed, at least in part, by a system for providing endovascular thermal ablation, such system 100 shown in FIG. 1. In some embodiments, the method shown in flow chart 700 may be performed, at least in part, by a power routing circuit, such as power routing circuit 330 and/or power routing circuit components 500, and/or 600.

At block 702, “applying a voltage in a first polarity between a first terminal and a second terminal” is performed. In some embodiments, the voltage may be provided by energy delivery console 104. The voltage may be provided to terminals A and B as shown in FIGS. 5 and 6.

At block 704, “passing a current through a first transistor responsive to the voltage” is performed. The current is passed responsive to the voltage increasing a gate-source voltage of the first transistor. The voltage may be an absolute value of the gate-source voltage in some embodiments. The transistor may be transistor 542 in some embodiments.

At block 706, “heating the first heating element responsive to the passing current” is performed. In some embodiments, the first heating element may be heating element 223.

At block 708, “impeding heating of the second heating element with a second transistor”is performed. In some embodiments, the second transistor may be transistor 544.

In one or more embodiments, the method shown in flow chart 700 may include additional steps. At block 710, “applying a second voltage in a second polarity opposite the first polarity between the first terminal and the second terminal” may be performed. At block 712, “passing a second current through the second transistor responsive to the second voltage” may be performed. The second current is passed responsive to the second voltage increasing a gate-source voltage of the second transistor in some embodiments. At block 714, “heating the first heating element and a second heating element responsive to the passing current” may be performed. In some embodiments, the second heating element may be heating element 225.

In some embodiments, the method shown in flow chart 700 may also include additional steps. At block 716, “passing the current through a first diode coupled in parallel with the first resistor responsive to the voltage” may be performed. The current is passed responsive to the voltage meeting or exceeding a forward voltage drop of the first diode. In some embodiments, the first diode may be diode 440. At block 718, “impeding heating of the second heating element with a second diode” may be performed. In some embodiments, the second diode may be diode 338.

In some embodiments, the method shown in flow chart 700 may further include block 718 where “passing the second current through a second diode responsive to the second voltage” may be performed. The second current is passed responsive to the voltage meeting or exceeding a forward voltage drop of the second diode.

The method shown in flow chart 700 may allow for one or both heating elements to be heated by passing a current through one or both heating elements by applying a voltage in a first or second polarity. When the voltage energizing the heaters is low, the transistors are not conducting, and the current is flowing through one of the diodes depending on voltage polarity. The method shown in flow chart 700 may reduce heat dissipation by power routing circuitry, which may reduce the effects on a temperature sensor located near the power routing circuitry (e.g., both located in a handle of a heated catheter for thermal ablation).

Power routing circuits as disclosed herein may include components with reduced forward voltage drops may be included in a handle of a thermal ablation catheter. In some examples, the power routing circuit may include transistors such as MOSFETs. In some examples, transistors may be provided in parallel with diodes in the power routing circuit. In some examples, the diodes may be Schottky diodes. The reduction in forward voltage drops may reduce heat dissipation by the power routing circuit, which may reduce heating of the reference temperature sensor (e.g., cold junction). This may improve accuracy of temperature measurements at the distal end of the catheter, helping to ensure effective treatment.

While the examples provided herein describe selecting between two heating elements, additional heating elements may be included, and the lower heat dissipation power routing components may be used. Further, while the example applications disclosed herein relate to endovascular ablation (e.g., venous ablation), the principles of the present disclosure are not so limited and may be applied to other heated catheter applications (e.g., thermal ablation of other tissues).

From the foregoing it will be appreciated that, although specific embodiments of the disclosure have been described herein for purposes of illustration, various modifications may be made without deviating from the spirit and scope of the disclosure. Accordingly, the disclosure is not limited except as by the appended claims.

What is claimed is:

1. A system for venous ablation, the system comprising:
a catheter including:
a tube including a first heating element and a second heating element coupled in series between a first terminal and a second terminal; and
a power routing circuit including: a first transistor having a first node coupled between the first heating element and the second heating element and a second node coupled to the first terminal of the catheter and a second transistor having a third node coupled to the second heating element and a fourth node coupled to the first terminal of the catheter, wherein the first transistor includes a different type than the second transistor.

2. The system of claim 1, wherein the first transistor includes an N-channel metal-oxide semiconductor field-effect transistor (MOSFET) and the second transistor includes a P-channel MOSFET.

3. The system of claim 1, wherein a gate of the first transistor and a gate of the second transistor are coupled to the second terminal.

4. The system of claim 1, further comprising a first diode coupled in parallel with the first transistor and a second diode coupled in parallel with the second transistor, wherein the first diode is configured to conduct in a first direction and the second diode is configured to conduct in a second direction different than the first direction.

5. The system of claim 4, wherein the first diode and the second diode each includes a Schottky diode.

6. The system of claim 3, further comprising a resistive divider coupled between the second terminal and the gates of the first and second transistors.

7. The system of claim 1, wherein the catheter further includes a handle coupled to the tube, wherein the power routing circuit is included in the handle.

8. The system of claim 7, wherein the tube further includes a first temperature sensor, and the handle includes a second temperature sensor.

9. The system of claim 1, further comprising an energy delivery console configured to provide a voltage to the first terminal and the second terminal.

10. A catheter for venous ablation, the catheter comprising:
a first transistor having a first node coupled between a first resistance and a second resistance and a second node coupled to a first terminal; and
a second transistor having a third node coupled to the second resistance and a fourth node coupled to the first terminal, wherein the first transistor includes a different type than the second transistor.

11. The catheter of claim 10, further comprising:
a first diode coupled in parallel with the first transistor; and
a second diode coupled in parallel with the second transistor.

12. The catheter of claim 11, wherein the first diode is configured to conduct in a first direction and the second diode is configured to conduct in a second direction different than the first direction.

13. The catheter of claim 10, wherein the first resistance and the second resistance each include a heating element.

14. The catheter of claim 10, wherein the first terminal and a second terminal are configured to selectively provide a voltage in a first polarity or a second polarity opposite the first polarity.

15. The catheter of claim 10, wherein the first transistor and the second transistor each include a MOSFET.

16. The catheter of claim 15, wherein the first transistor includes an N-channel MOSFET and the second transistor includes a P-channel MOSFET.

17. A method of controlling first and second heating elements of a system for venous ablation, the method comprising:
applying a voltage in a first polarity between a first terminal and a second terminal;
passing a current through a first transistor responsive to the voltage, wherein the current is passed responsive to the voltage increasing a gate-source voltage of the first transistor;
heating the first heating element responsive to the passing current; and
impeding heating of the second heating element with a second transistor.

18. The method of claim 17, further comprising:
applying a second voltage in a second polarity opposite the first polarity between the first terminal and the second terminal;
passing a second current through the second transistor responsive to the second voltage, wherein the second current is passed responsive to the second voltage increasing a gate-source voltage of the second transistor; and
heating the first heating element and a second heating element responsive to the passing current.

19. The method of claim 17, further comprising:
passing the current through a first diode coupled in parallel with the first resistor responsive to the voltage, wherein the current is passed responsive to the voltage meeting or exceeding a forward voltage drop of the first diode; and
impeding heating of the second heating element with a second diode.

20. The method of claim 19, further comprising:
applying a second voltage in a second polarity opposite the first polarity between the first terminal and the second terminal;
passing a second current through the second transistor responsive to the second voltage, wherein the second current is passed responsive to the second voltage increasing a gate-source voltage of the second transistor; and
passing the second current through a second diode responsive to the second voltage, wherein the second current is passed responsive to the voltage meeting or exceeding a forward voltage drop of the second diode.
